(19) United States
(12) Patent Application Publication
(10) Pub. No.: US 2026/0020752 A1
(43) Pub. Date: Jan. 22, 2026

(54) MEDICAL SUPPORT DEVICE, MEDICAL SUPPORT METHOD, AND MEDICAL SUPPORT PROGRAM

(71) Applicant: FUJIFILM Corporation, Tokyo (JP)

(72) Inventor: Yoshiro KITAMURA, Tokyo (JP)

(73) Assignee: FUJIFILM Corporation, Tokyo (JP)

(21) Appl. No.: 19/260,619

(22) Filed: Jul. 7, 2025

(30) Foreign Application Priority Data
Jul. 16, 2024 (JP) 2024-113673

(51) Int. Cl. A61B 1/04; A61B 1/00

(52) U.S. Cl. CPC A61B 1/042; A61B 1/00009; A61B 1/00045; A61B 1/00087

(57) ABSTRACT

A medical support device including a processor, wherein the processor is configured to: acquire a first image and a second image by optically imaging a surgical field including a target part inside a body from different viewpoints using a first camera and a second camera; detect a marker composed of an optically detectable pattern disposed in the surgical field from at least the first image; and generate a three-dimensional model showing a surface shape of the target part based on a relative positional relationship between the viewpoint of the first camera and the viewpoint of the second camera, the first image, and the second image, the relative positional relationship being specified based on the marker.

### CROSS-REFERENCE TO RELATED APPLICATIONS

This application claims priority from Japanese Application No. 2024-113673, filed on Jul. 16, 2024, the entire disclosure of which is incorporated herein by reference.

### BACKGROUND

### Technical Field

The present disclosure relates to a medical support device, a medical support method, and a medical support program.

### Related Art

In the related art, a technology of specifying a position and an orientation of a medical instrument inserted into a body and supporting medical care such as a surgery and an examination based on the specified position and orientation of the medical instrument is known. For example, WO2023/162657A discloses a method of estimating a position and an orientation of an ultrasound probe by performing image analysis on a surgical field image obtained by optically imaging a surgical field including the ultrasound probe, and superimposing and displaying assistive information corresponding to the estimated position and orientation on the surgical field image.

In addition, for example, JP2021-085751A discloses a method of acquiring a first image and a second image by imaging a marker provided on an object from different viewpoints, detecting a region of the marker from each of the first image and the second image, and acquiring three-dimensional coordinates of the marker using a stereo method. In addition, for example, in “Stoyanov, et. al., “Dense 3D Depth Recovery for Soft Tissue Deformation During Robotically Assisted Laparoscopic Surgery”, Medical Image Computing and Computer-Assisted Intervention (MICCAI) 2004.”, a method of attaching two cameras to a distal end of a rigid endoscope to obtain a parallax picture, recognizing a depth of an organ surface using a stereo method, and reconstructing a three-dimensional model of the organ surface is disclosed.

In an endoscopic surgery, an internal structure is grasped while adjusting a position and an orientation of an ultrasound probe in a surgical field by observing a surgical field image optically captured by an endoscope and an ultrasound image. However, since the surgical field image and the ultrasound image have different viewpoints, imaging ranges, drawing methods, and the like, it is difficult to appropriately adjust the position and the orientation of the ultrasound probe by comparing the surgical field image and the ultrasound image, and a high level of skill is required.

### SUMMARY

The present disclosure provides a medical support device, a medical support method, and a medical support program capable of supporting observation of an internal image.

According to a first aspect of the present disclosure, there is provided a medical support device comprising: a processor, in which the processor is configured to acquire a first image and a second image by optically imaging a surgical field including a target part inside a body from different viewpoints using a first camera and a second camera, detect a marker composed of an optically detectable pattern disposed in the surgical field from at least the first image, and generate a three-dimensional model showing a surface shape of the target part based on a relative positional relationship between the viewpoint of the first camera and the viewpoint of the second camera, the first image, and the second image, the relative positional relationship being specified based on the marker.

The first camera may be a camera provided on a trocar for inserting a medical instrument into the body or on a body wall inside the body.

The second camera may be a camera provided at a distal end part of an endoscope used in an endoscopic surgery.

The marker may be attached to an outer peripheral surface of the second camera and is imaged by the first camera, and the processor may be configured to specify the relative positional relationship between the viewpoint of the first camera and the viewpoint of the second camera based on the marker in the first image.

The marker may be attached to a medical instrument to be inserted into the body and is imaged by each of the first camera and the second camera, and the processor may be configured to specify the relative positional relationship between the viewpoint of the first camera and the viewpoint of the second camera based on the marker in the first image and the marker in the second image.

The medical instrument may be an ultrasound probe that transmits an ultrasound wave to the target part and that detects an electric signal corresponding to an ultrasound echo reflected from the target part.

The processor may be configured to acquire an internal image showing an internal structure of the target part, acquire position and orientation information indicating a position and an orientation of a medical instrument inserted into the body, and control display of a superimposed image that is obtained by superimposing the internal image on the three-dimensional model and in which a display aspect of the internal image is adjusted based on the position and orientation information.

According to a second aspect of the present disclosure, there is provided a medical support method executed by a computer, the medical support method comprising: acquiring a first image and a second image by optically imaging a surgical field including a target part inside a body from different viewpoints using a first camera and a second camera; detecting a marker composed of an optically detectable pattern disposed in the surgical field from at least the first image; and generating a three-dimensional model showing a surface shape of the target part based on a relative positional relationship between the viewpoint of the first camera and the viewpoint of the second camera, the first image, and the second image, the relative positional relationship being specified based on the marker.

According to a third aspect of the present disclosure, there is provided a medical support program causing a computer to execute: acquiring a first image and a second image by optically imaging a surgical field including a target part inside a body from different viewpoints using a first camera and a second camera; detecting a marker composed of an optically detectable pattern disposed in the surgical field from at least the first image; and generating a three-dimensional model showing a surface shape of the target part based on a relative positional relationship between the viewpoint of the first camera and the viewpoint of the second camera, the first image, and the second image, the relative positional relationship being specified based on the marker.

According to the above aspects, a medical support device, a medical support method, and a medical support program of the present disclosure can support observation of an internal image.

### BRIEF DESCRIPTION OF THE DRAWINGS

FIG. 1 is a diagram showing an outline of a medical support system including a medical support device.

FIG. 2 is a diagram showing a state of an inside of a body in an endoscopic surgery.

FIG. 3 is a diagram showing an insertion state of a biopsy needle guided by a guide groove.

FIG. 4 is a diagram showing an example of a hardware configuration of the medical support device.

FIG. 5 is a diagram showing an example of a functional configuration of the medical support device.

FIG. 6 is a diagram showing a relationship in a position and an orientation between a marker and an ultrasound probe.

FIG. 7 is a diagram showing an example of a superimposed image.

FIG. 8 is a diagram showing a relationship in a position and an orientation between a marker and an ultrasound probe.

FIG. 9 is a diagram showing an example of a superimposed image.

FIG. 10 is a diagram showing an example of a three-dimensional superimposed image.

FIG. 11 is a diagram showing an example of a three-dimensional superimposed image.

FIG. 12 is a diagram showing a relationship in a position and an orientation between a marker and an ultrasound probe.

FIG. 13 is a diagram showing an example of a superimposed image.

FIG. 14 is a diagram showing an example of a three-dimensional superimposed image.

FIG. 15 is a diagram showing an example of a three-dimensional superimposed image.

FIG. 16 is a diagram for describing an example of a method of generating a three-dimensional model.

FIG. 17 is a diagram for describing another example of a method of generating a three-dimensional model.

FIG. 18 is a flowchart showing an example of medical support processing.

FIG. 19 is a flowchart showing an example of three-dimensional model generation processing.

### DETAILED DESCRIPTION

Hereinafter, an example of an embodiment of the disclosed technology will be described with reference to the drawings. The same or equivalent components and parts in the respective drawings are denoted by the same reference numerals, and the duplicated description will be omitted. In addition, dimensional ratios in the drawings are exaggerated for convenience of description and may be different from the actual ratios.

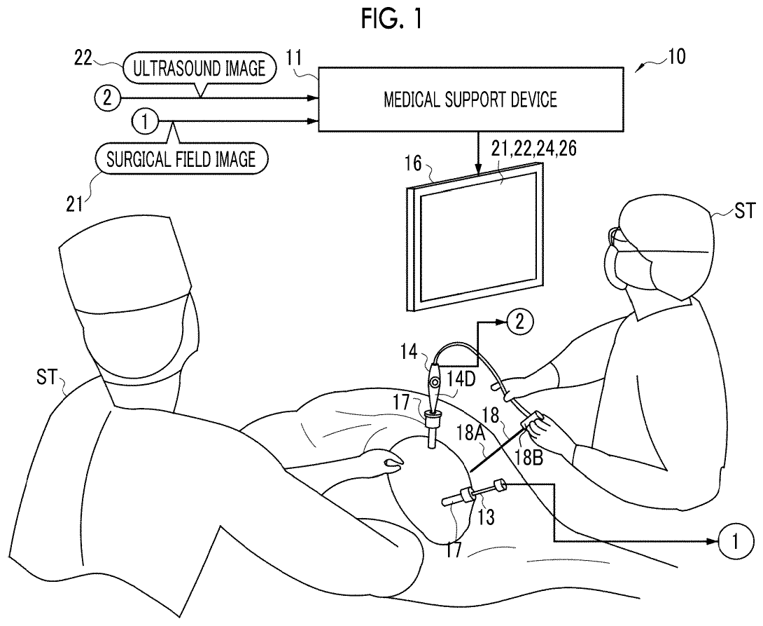

An example of a medical support system 10 to which a medical support device 11 according to the present embodiment is applied will be described with reference to FIGS. 1 to 3. As an example, the medical support system 10 is used in a case in which an endoscopic surgery using an endoscope 13 is performed on a patient PT. The endoscopic surgery is a surgery that is performed by making a small hole in a body of the patient PT and inserting a medical instrument such as the endoscope 13 through the hole, unlike a laparotomy. The medical support system 10 provides a medical staff ST including a doctor with a visual field of a surgical field inside the body of the patient PT, and with support information for supporting medical care such as a surgery and an examination. The support information, as described below, includes a superimposed image 26 obtained by superimposing and displaying an ultrasound image 22 on a surgical field image 21. Such a medical support system 10 has a function of providing the support information in real time during a surgery, and is therefore also called a surgical navigation system or the like.

As shown in FIG. 1, the medical support system 10 comprises a medical support device 11, an endoscope 13, an ultrasound probe 14, and a display 16. The medical support device 11 is communicably connected to the endoscope 13, the ultrasound probe 14, and the display 16.

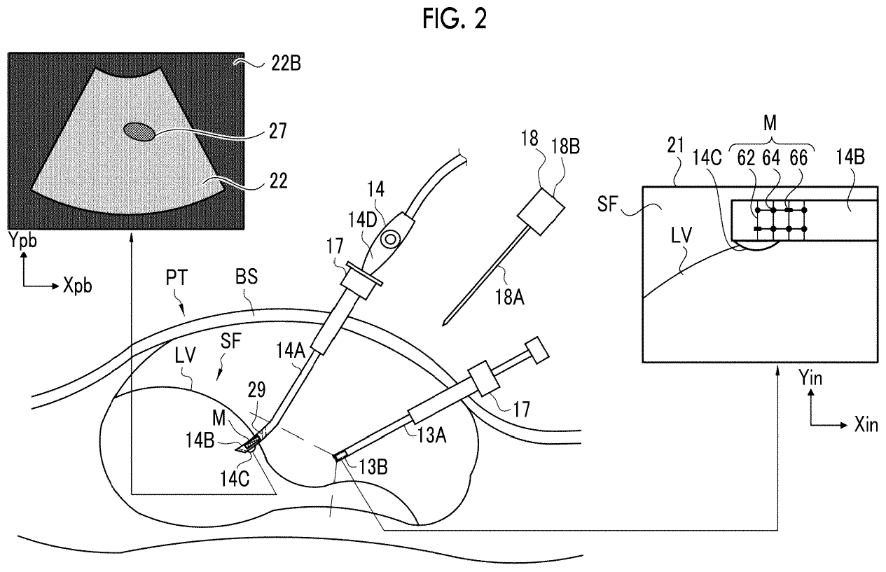

FIG. 2 shows a state in which the endoscope 13 and the ultrasound probe 14 are inserted into the abdomen of the patient PT. In the endoscopic surgery, a part of the endoscope 13 and a part of the ultrasound probe 14 including distal end parts thereof are inserted into the body via a trocar 17. The trocar 17 is an insertion tool having an insertion hole into which the endoscope 13 or the like is inserted and a valve provided in the insertion hole to prevent gas leakage. In the endoscopic surgery, the trocar 17 is used for insertion into the body, such as the endoscope 13 and the ultrasound probe 14, because pneumoperitoneum is performed by injecting carbon dioxide gas into an abdominal cavity.

The biopsy needle 18 is a treatment tool for puncturing a lesion such as a tumor included in an organ as a puncture target. Specifically, the biopsy needle 18 has a needle part 18A and a grip part 18B provided on a base end side of the needle part 18A. The biopsy needle 18 is, for example, a cauterization biopsy needle used for cauterization of the lesion. The cauterization biopsy needle has an electrode at its distal end to which a high-frequency voltage is applied, and, in a case in which the high-frequency voltage is applied in a state in which the lesion is punctured by the electrode, the lesion is necrotized by heat generated by the electrode. In the present embodiment, as an example, an example will be described in which a treatment of necrotizing a tumor 27 of a liver LV is performed by visualizing the tumor 27 by using an ultrasound image 22 and cauterizing the visualized tumor 27 by using the biopsy needle 18.

The endoscope 13 optically images a surgical field SF including a target part (in this example, the liver LV) inside the body of the patient PT by using the camera 13B. The surgical field SF is a space that spreads in a body cavity defined by an organ and a body wall inside the body. Specifically, the endoscope 13 has an insertion part 13A to be inserted into the body of the patient PT. A camera 13B and a light source (for example, a light emitting diode (LED)) for illumination are incorporated in a distal end part of the insertion part 13A. The endoscope 13 is, for example, a rigid endoscope in which the insertion part 13A is rigid, and is often used for abdominal cavity observation, so that the endoscope 13 is also called a laparoscope.

The camera 13B has an image sensor such as a charge coupled device (CCD) image sensor and a complementary metal oxide semiconductor (CMOS) image sensor, and an imaging optical system including a lens that forms a subject image on an imaging surface of the image sensor. The image sensor is, for example, an image sensor capable of capturing a color image. The endoscope 13 is connected to an image processing processor for an endoscope (not shown). This image processing processor performs signal processing on an imaging signal output by the image sensor to generate a surgical field image 21 of the surgical field SF inside the body. The surgical field image 21 captured by the endoscope 13 is transmitted to the medical support device 11 in real time via the image processing processor for an endoscope. In FIG. 2, reference numerals Xin and Yin indicate a coordinate system of the surgical field image 21. The camera 13B is an example of a “camera” and a “camera provided at a distal end part of an endoscope used in an endoscopic surgery” of the present disclosure.

As illumination light for the endoscope 13, for example, visible light such as white light is used. As the illumination light of the endoscope 13, special light such as ultraviolet light and infrared light may be used. As the special light, for example, light restricted to a specific wavelength such as short-wavelength narrow-band light obtained by narrowing down light in a short wavelength range such as an ultraviolet range may be used. The surgical field image 21 is a video of the surgical field SF illuminated with illumination light, and more specifically, is a video based on light reflected from the illumination light near the surface of the surgical field SF. Therefore, in the surgical field image 21, a structure present in the vicinity of a surface layer of the target part can be visualized, but it is difficult to observe an internal structure.

The ultrasound probe 14 transmits an ultrasound wave to the target part and detects an electric signal corresponding to an ultrasound echo reflected from the target part. Specifically, the ultrasound probe 14 has an insertion part 14A to be inserted into the body of the patient PT and an operation part 14D on a base end side of the insertion part 14A. An ultrasound transducer 14C is incorporated in a distal end part 14B of the insertion part 14A. The ultrasound probe 14 is an example of a “medical instrument” of the present disclosure.

The ultrasound transducer 14C transmits an ultrasound wave to the target part and receives an ultrasound echo reflected from the target part. The ultrasound probe 14 is connected to an image processing processor for an ultrasound probe (not shown). This image processing processor performs image reconstruction processing based on an electric signal corresponding to the ultrasound echo received by the ultrasound transducer 14C. Through the image reconstruction processing, the ultrasound image 22 showing an internal structure of the target part scanned by the ultrasound probe 14 is generated. The ultrasound image 22 captured by the ultrasound probe 14 is transmitted to the medical support device 11 in real time via the image processing processor for an ultrasound probe. In FIG. 2, reference numerals Xpb and Ypb indicate a coordinate system of the ultrasound image 22.

The ultrasound probe 14 is, for example, a convex type that radially transmits ultrasound waves, and acquires a fan-shaped ultrasound image 22 with the ultrasound transducer 14C as a base point. A plurality of the ultrasound images 22 are captured along a scanning direction by performing the scanning with the ultrasound probe 14. In addition, the ultrasound image 22 is a so-called brightness (B)-mode image in which an internal structure from a surface layer to a deep layer where the ultrasound wave reaches the target part is visualized as brightness information. The ultrasound image 22 visualizes an internal structure of the target part that cannot be observed in the surgical field image 21 obtained by optical imaging.

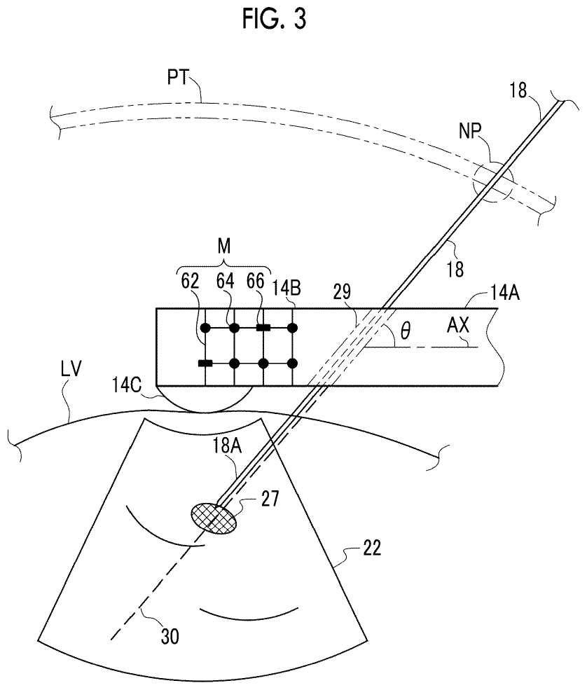

A guide groove 29 is provided in the distal end part 14B of the insertion part 14A. The guide groove 29 is a guide groove for engaging with the biopsy needle 18 to guide the insertion of the biopsy needle 18 to a target position inside the organ. FIG. 3 schematically shows a state in which the biopsy needle 18 inserted into the body from an insertion position NP on a body surface BS of the patient PT punctures the tumor 27 in the liver LV while being guided by the guide groove 29. The guide groove 29 is provided, for example, on the base end side with respect to the ultrasound transducer 14C in the distal end part 14B, and is inclined at an angle θ with respect to a direction of an axis AX of the distal end part 14B. The first guide groove 29 is inclined rearward such that a needle tip of the biopsy needle 18 to be inserted from the base end side of the distal end part 14B is directed toward the distal end side of the distal end part 14B.

The biopsy needle 18 is inserted while the tumor 27 is checked by the ultrasound image 22. Since a region visualized by the ultrasound image 22 is a radial region with the ultrasound transducer 14C as a base point, the inclination of the guide groove 29 makes it possible to direct the needle tip of the biopsy needle 18 toward the tumor 27 visualized by the ultrasound image 22. The insertion part 14A may be provided with a plurality of guide grooves 29 having different angles with respect to the direction of the axis AX. In this case, the medical staff ST can select an appropriate guide groove 29 and insert the biopsy needle 18.

In addition, a marker M is provided to an outer peripheral surface of the distal end part 14B of the insertion part 14A. The marker M is a marker that is recognizable from the surgical field image 21 optically captured by the camera 13B of the endoscope 13, that is, an optically detectable marker. The marker M is used by the medical support device 11 to estimate a position and an orientation of the ultrasound probe 14, more specifically, a position and an orientation of the distal end part 14B of the insertion part 14A in the surgical field SF. A method of estimating the position and the orientation using the marker M will be described below.

As an example, the marker M is a marker of a lattice pattern 62 composed of a first line extending in the direction of the axis AX of the distal end part 14B of the ultrasound probe 14 and a second line orthogonal to the axial direction of the distal end part 14B and formed in the circumferential direction along an outer peripheral surface of the distal end part 14B. In addition, a circular symbol 64 or a rectangular symbol 66 is assigned to each intersection in the lattice pattern 62.

The marker M need only include at least the symbols 64 and 66 disposed at each intersection of the lattice pattern 62, and line segments indicating the first line and the second line as shown in FIG. 3 and the like does not need to be included. In addition, the symbols 64 and 66 on the marker M can be any shape, not limited to a circle and a rectangle, and may be represented by figures such as a triangle, a polygon, a star shape, and various marks, or may be represented by a character or the like. In addition, in each drawing, in order to avoid complication of the drawing, the marker M may be simplified or omitted.

The medical support device 11 acquires the surgical field image 21 from the endoscope 13, and acquires the ultrasound image 22 from the ultrasound probe 14. In addition, the medical support device 11 generates a superimposed image 26 by superimposing the ultrasound image 22 on the surgical field image 21, and controls the display 16 to display the superimposed image 26. The superimposed image 26 displayed on the display 16 provides the medical staff ST with the visual field of the surgical field SF inside the body of the patient PT and the internal structure of the target part (liver LV).

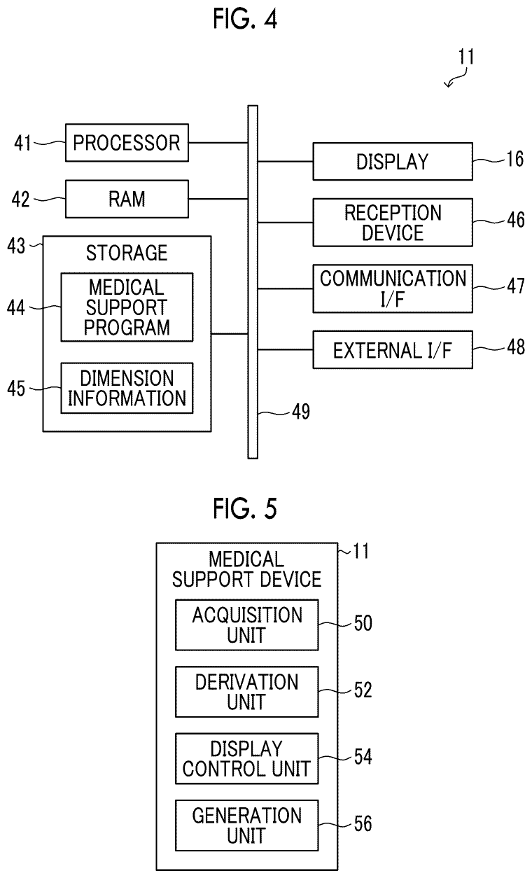

Next, a configuration of the medical support device 11 will be described. FIG. 4 shows an example of a hardware configuration of the medical support device 11. The medical support device 11 comprises a display 16, a processor 41, a random access memory (RAM) 42, a storage 43, a reception device 46, a communication interface (I/F) 47, and an external I/F 48. These units are connected to a bus 49 such as a system bus and a control bus, and can communicate with each other.

In addition to the superimposed image 26, various types of information are displayed on the display 16. Examples of the display 16 include a liquid crystal display and an electro-luminescence (EL) display. The number of the displays 16 need only be at least one as shown in FIG. 1, and may be more than one.

The processor 41 is, for example, a central processing unit (CPU), and integrally controls the respective units of the medical support device 11 in accordance with a control program and executes various types of processing in accordance with various types of application programs.

The RAM 42 is a memory that transitorily stores information, and is used as a work memory by the processor 41. Examples of the RAM 42 include a dynamic random access memory (DRAM) and a static random access memory (SRAM).

The storage 43 is a non-volatile storage device that stores various programs, various parameters, and the like. Examples of the storage 43 include a hard disk drive (HDD) and a solid state drive (SSD). The storage 43 stores a medical support program 44 for causing a computer to function as the medical support device 11.

In addition, the storage 43 stores dimension information 45. The dimension information 45 includes information indicating dimensions of the ultrasound probe 14, specifically, a relative positional relationship between the marker M, the ultrasound transducer 14C, the guide groove 29, and the like in the ultrasound probe 14. The positional relationship of the marker M is, for example, information on the position and the orientation at which the symbols 64 and 66 constituting the marker M are provided with respect to the axial direction and the circumferential direction of the distal end part 14B.

The positional relationship of the ultrasound transducer 14C is represented by a linear distance between a reference point of the distal end part 14B and the ultrasound transducer 14C, an inclination angle of the ultrasound transducer 14C with respect to the direction of the axis AX of the distal end part 14B, and the like. The positional relationship of the guide groove 29 is represented by a linear distance between the reference point of the distal end part 14B and the guide groove 29, an inclination angle θ of the guide groove 29 with respect to the direction of the axis AX of the distal end part 14B, and the like.

The reception device 46 includes a keyboard, a mouse, and the like (not shown), and receives an instruction from the operator. That is, the medical support device 11 is operated by an operator such as the medical staff ST through the reception device 46. The reception device 46 may be a device that receives touch input, such as a touch panel, a device that receives voice input, such as a microphone, a device that receives gesture input, such as a camera, or the like.

The communication I/F 47 is connected to a network (not shown) such as a local area network (LAN) and/or a wide area network (WAN), and performs transmission control in accordance with a communication protocol defined in various types of wired or wireless communication standards.

The external I/F 48 is, for example, a universal serial bus (USB) interface, and is used for connection to peripheral devices such as a printer and a memory card. As the medical support device 11, for example, a personal computer, a server computer, a smartphone, a tablet terminal, or a wearable terminal can be applied as appropriate.

Next, a functional configuration of the medical support device 11 will be described. FIG. 5 is a block diagram showing an example of the functional configuration of the medical support device 11. The medical support device 11 comprises an acquisition unit 50, a derivation unit 52, a display control unit 54, and a generation unit 56. The processor 41 reads out the medical support program 44 from the storage 43 and executes the medical support program 44 on the RAM 42, so that the processor 41 functions as each functional unit of the acquisition unit 50, the derivation unit 52, the display control unit 54, and the generation unit 56. The processor 41 operates as each functional unit of the acquisition unit 50, the derivation unit 52, the display control unit 54, and the generation unit 56 to implement the medical support processing.

The acquisition unit 50 acquires the surgical field image 21 obtained by optically imaging the surgical field SF including the target part inside the body and the medical instrument inserted into the body using the camera 13B. For example, the acquisition unit 50 acquires the surgical field image 21 from a device including a processor of the endoscope 13 via the external I/F 48 or the communication I/F 47. As shown in FIG. 2, in a case in which the ultrasound probe 14 is inserted into the surgical field SF, the ultrasound probe 14, more specifically, the distal end part 14B of the insertion part 14A is reflected in the surgical field image 21.

In addition, the acquisition unit 50 acquires an internal image showing the internal structure of the target part. For example, the acquisition unit 50 acquires the ultrasound image 22 from the device including the processor of the ultrasound probe 14 via the external I/F 48 or the communication I/F 47. The ultrasound image 22 is an example of an “internal image” of the present disclosure. The medical support device 11 may have the processor of the endoscope 13 and/or the processor of the ultrasound probe 14.

The derivation unit 52 derives position and orientation information indicating the position and the orientation of the medical instrument (for example, the ultrasound probe 14) in the surgical field SF based on the surgical field image 21, more specifically, the marker M included in the surgical field image 21. Specifically, the derivation unit 52 detects the marker M by searching for morphological features of the marker M, such as the symbols 64 and 66, from the surgical field image 21. For example, the derivation unit 52 may detect the marker M using an image processing method such as pattern matching.

In addition, for example, the marker M may be detected using an artificial intelligence (AI) technology using a machine learning model instead of a rule-based method such as pattern matching. As such a machine learning model, for example, a neural network model such as a convolutional neural network (CNN) that has been trained in advance by using the surgical field image 21 as an input and a region of the marker M in the input surgical field image 21 as an output can be applied.

The display control unit 54 controls the display of the superimposed image 26 that is obtained by superimposing the internal image (for example, the ultrasound image 22) on the surgical field image 21 and in which a display aspect of the internal image is adjusted based on the position and orientation information. As an example, the display control unit 54 generates the superimposed image 26 showing an imaging range (for example, the position, the orientation, and the size) of the ultrasound image 22 in the surgical field SF by superimposing the ultrasound image 22 on a region on the surgical field image 21 corresponding to the imaging range of the ultrasound image 22. The display aspect is a position and an orientation in a case in which the internal image is displayed on the superimposed image 26, the presence or absence of the display, and the like. The superimposed image 26 is an example of a “second superimposed image” of the present disclosure.

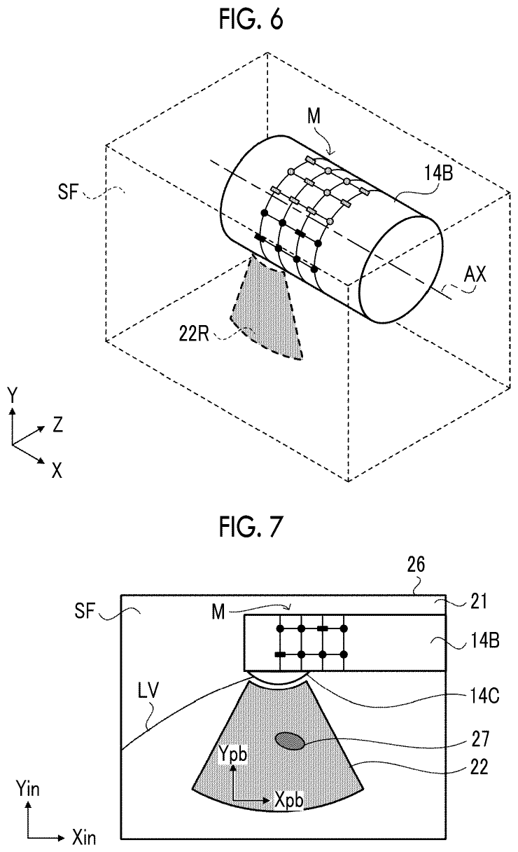

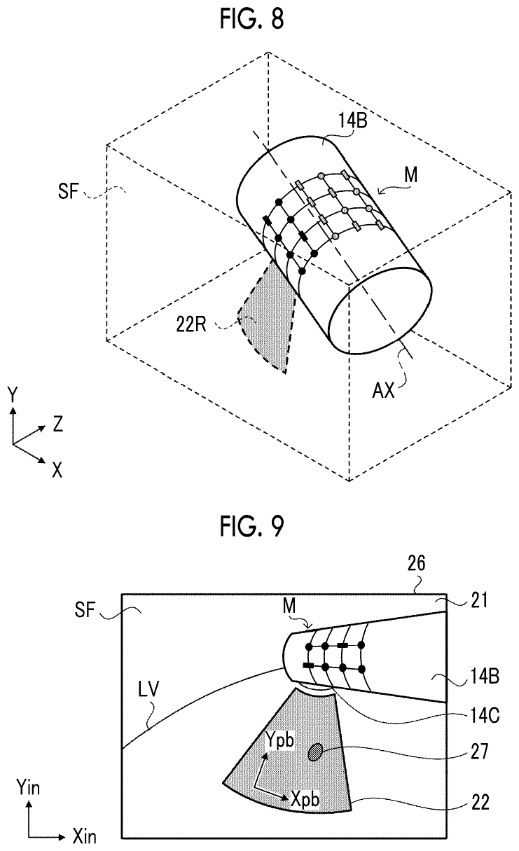

A specific example of the superimposed image 26 will be described with reference to FIGS. 6 to 9. FIGS. 6 and 8 are diagrams conceptually showing the position and the orientation of the distal end part 14B of the ultrasound probe 14 in the surgical field SF defined as a three-dimensional space, each of which assumes a different position and orientation. FIGS. 7 and 9 show an example of the superimposed image 26 generated according to the position and the orientation of the ultrasound probe 14 in the surgical field SF. FIG. 7 is a superimposed image 26 in which the position and the orientation of the distal end part 14B of the ultrasound probe 14 are displayed in the state of FIG. 6. FIG. 9 is a superimposed image 26 in which the position and the orientation of the distal end part 14B of the ultrasound probe 14 are displayed in the state of FIG. 8.

In the surgical field SF of the three-dimensional space shown in FIGS. 6 and 8, the Z-axis is a direction parallel to an imaging optical axis of the camera 13B of the endoscope 13. In addition, in the surgical field SF of the three-dimensional space, an X-Y plane is a plane parallel to an imaging surface of the camera 13B and is orthogonal to the imaging optical axis. That is, the X-Y plane of the surgical field SF, which is a three-dimensional space, is parallel to a screen (Xin-Yin plane) of the surgical field image 21. The surgical field image 21 is a projection image obtained by projecting the surgical field SF from one viewpoint. In FIGS. 6 and 8, among the symbols 64 and 66 constituting the marker M, a symbol reflected in the surgical field image 21 is shown in a dark color, and a symbol not reflected in the surgical field image 21 is shown in a light color.

FIG. 6 shows a state in which the direction of the axis AX of the distal end part 14B of the ultrasound probe 14 is orthogonal to the imaging optical axis of the camera 13B in the surgical field SF of the three-dimensional space (more specifically, a state in which the axis AX of the distal end part 14B is parallel to the X-axis). In this case, as shown in FIG. 7, the marker M reflected in the surgical field image 21 has orthogonal lines of the lattice pattern 62 parallel to the X-axis and Y-axis. In addition, the symbols 64 and 66 are reflected in the surgical field image 21 at equal intervals.

On the other hand, FIG. 8 shows a state in which the direction of the axis AX of the distal end part 14B of the ultrasound probe 14 is not orthogonal to the imaging optical axis of the camera 13B and is inclined in a depth direction parallel to the imaging optical axis in the surgical field SF of the three-dimensional space. The orientation shown in FIG. 8 is a state in which the axis AX of the distal end part 14B is rotated by approximately-25 degrees about the Y-axis from the orientation shown in FIG. 6. In this case, as shown in FIG. 9, the marker M reflected in the surgical field image 21 is reflected such that the farther the marker M is from the camera 13B in the depth direction, the shorter the line extending in the circumferential direction among the lines of the lattice pattern 62 is, and the shorter the interval between the symbols 64 and 66 is.

In this way, the form of the marker M reflected in the surgical field image 21 changes depending on the orientation of the distal end part 14B. The derivation unit 52 estimates the orientation of the distal end part 14B of the ultrasound probe 14 in the surgical field SF based on the form of the marker M in the surgical field image 21. Specifically, the derivation unit 52 detects the direction of the axis AX of the distal end part 14B in the surgical field SF as the orientation of the distal end part 14B.

In addition, in a case in which the position of the distal end part 14B is changed in the surgical field SF, the position of the marker M reflected in the surgical field image 21 is also changed. The derivation unit 52 estimates the position of the distal end part 14B in the surgical field SF based on the position of the marker M. The position of the distal end part 14B is detected as a position of a reference point of the distal end part 14B, which is provided at, for example, a distal end position of the distal end part 14B. Further, an imaging distance from the camera 13B to the marker M in the surgical field SF (that is, a distance in the Z-axis direction parallel to the imaging optical axis) can be calculated based on a focal length of the camera 13B and the size of the marker M reflected in the surgical field image 21. The derivation unit 52 derives position coordinates of the reference point of the distal end part 14B in the surgical field SF based on the dimension information 45 of the distal end part 14B, which includes the imaging distance and the known dimensions of the marker M.

In the example of FIG. 6, the position of the distal end part 14B of the ultrasound probe 14 is estimated as information such as the position coordinates (X01, Y01, Z01) of the reference point of the distal end part 14B in the surgical field SF. The orientation of the distal end part 14B is defined as the direction of the axis AX of the distal end part 14B in the surgical field SF. For example, the axis AX of the distal end part 14B is estimated as information indicating that it is parallel to the XY plane and the XZ plane and that is orthogonal to the YZ plane.

In the example of FIG. 8, the position of the distal end part 14B of the ultrasound probe 14 is estimated as information such as the position coordinates (X02, Y02, Z02) of the reference point of the distal end part 14B in the surgical field SF. The orientation of the distal end part 14B is defined as the direction of the axis AX of the distal end part 14B in the surgical field SF. For example, the axis AX of the distal end part 14B is estimated as information indicating that it is −25° with respect to the XY plane, parallel to the XZ plane, and is 65° with respect to the YZ plane.

In a case in which the position and the orientation of the distal end part 14B in the surgical field SF are estimated by the derivation unit 52, the display control unit 54 estimates the position and the orientation of the ultrasound transducer 14C in the surgical field SF based on the estimated position and orientation of the distal end part 14B and the dimension information 45. As described above, a linear distance between the reference point of the distal end part 14B and the ultrasound transducer 14C and the inclination angle of the ultrasound transducer 14C with respect to the direction of the axis AX of the distal end part 14B are known information defined as the dimension information 45. By using the dimension information 45, the position and the orientation of the ultrasound transducer 14C in the surgical field SF can be estimated based on the position and the orientation of the distal end part 14B in the surgical field SF.

In the example of FIG. 6, the position of the ultrasound transducer 14C is estimated as information such as the position coordinates (X11, Y11, Z11) obtained by correcting the position coordinates (X01, Y01, Z01) of the reference point of the distal end part 14B in the surgical field SF using the dimension information 45. The orientation of the ultrasound transducer 14C is defined as an orientation with respect to the axis AX of the distal end part 14B, which is a known inclination angle as the dimension information 45.

In the example of FIG. 8, the position of the ultrasound transducer 14C is estimated as information such as the position coordinates (X12, Y12, Z12) obtained by correcting the position coordinates (X02, Y02, Z02) of the reference point of the distal end part 14B in the surgical field SF using the dimension information 45. The orientation of the ultrasound transducer 14C is defined as an orientation with respect to the axis AX of the distal end part 14B, which is a known inclination angle as the dimension information 45.

In addition, the display control unit 54 estimates the imaging range of the ultrasound image 22 in the surgical field SF based on the estimated position and orientation of the ultrasound transducer 14C. The imaging range of the ultrasound image 22 corresponds to the position and the orientation of the ultrasound transducer 14C. Therefore, in a case in which the position and the orientation of the ultrasound transducer 14C in the surgical field SF are known, the imaging range of the ultrasound image 22 in the surgical field SF can also be estimated. For example, the imaging range of the ultrasound image 22 captured by the convex type ultrasound transducer 14C is a fan-shaped range that spreads radially from the ultrasound transducer 14C as a base point. In FIGS. 6 and 8, an imaging range 22R of the ultrasound image 22 in the surgical field SF is shown.

Then, the display control unit 54 generates the superimposed image 26 by superimposing the ultrasound image 22 on a region (Xin-Yin plane) in the surgical field image 21 corresponding to the imaging range (Xpb-Ypb plane) of the ultrasound image 22 in the surgical field SF. In the examples of FIGS. 6 and 7, the imaging range (Xpb-Ypb plane) of the ultrasound image 22 in the surgical field SF and the screen (Xin-Yin plane) of the surgical field image 21 in the surgical field SF are parallel to each other. On the other hand, in the examples of FIGS. 8 and 9, the imaging range (Xpb-Ypb plane) of the ultrasound image 22 in the surgical field SF and the screen (Xin-Yin plane) of the surgical field image 21 in the surgical field SF are not parallel to each other. The display control unit 54 performs deformation such as projective transformation, affine transformation, movement, rotation, enlargement, and reduction on the ultrasound image 22 in consideration of a difference in coordinate systems between the surgical field image 21 and the ultrasound image 22, and then generates the superimposed image 26 superimposed on the surgical field image 21.

With the superimposed image 26, for example, even in a case in which it is difficult to visually recognize the position and the orientation of the ultrasound transducer 14C in the surgical field image 21 due to shield by an organ or the like and a positional relationship with the camera 13B, the position and the orientation can be easily grasped. Therefore, it is easy to adjust the position and the orientation of the ultrasound probe 14 so that a desired region (for example, the tumor 27) is reflected in the ultrasound image 22, and it is possible to contribute to the improvement of the operability of the ultrasound probe 14.

As shown in FIG. 2, a margin region 22B may be added to the ultrasound image 22 to make the overall shape of the ultrasound image 22 rectangular. In this case, it is preferable that the display control unit 54 generates the superimposed image 26 by using an image from which the margin region 22B is removed as the internal image. As described above, in a case in which the ultrasound probe 14 is of the convex type, the ultrasound image 22 has a fan shape. The margin region 22B is provided to make the fan-shaped ultrasound image 22 rectangular for standardization of image processing. That is, information necessary for medical care, such as the internal structure of the target part in the ultrasound image 22, is not shown in the margin region 22B. By not including the margin region 22B in the superimposed image 26, it is possible to avoid difficulty in visual recognition of the surgical field image 21 in the superimposed image 26. FIG. 7 and the like show the superimposed image 26 from which the margin region 22B has been removed.

As described above, the superimposed image 26 is generated by using the surgical field image 21 captured by the endoscope 13. Since the surgical field image 21 is a projection image obtained by projecting the surgical field SF from one viewpoint, depth information is not included. Therefore, depending on the position and the orientation of the ultrasound probe 14, in the superimposed image 26 based on the surgical field image 21, a positional relationship (particularly, a front-rear relationship) between the target part in the surgical field SF and the imaging range of the ultrasound image 22 may be difficult to understand. In addition, depending on the position and the orientation of the ultrasound probe 14, it may be difficult to check the ultrasound image 22.

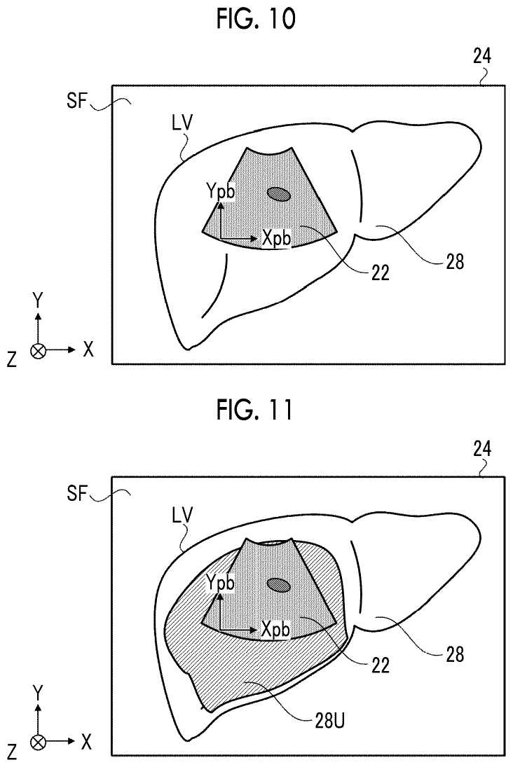

Therefore, in the medical support device 11 according to the present embodiment, as shown in FIG. 10, a three-dimensional superimposed image 24 obtained by superimposing the internal image (for example, the ultrasound image 22) on a three-dimensional model 28 showing the surface shape of the target part inside the body is generated. As a result, the positional relationship between the target part in the surgical field SF and the imaging range of the ultrasound image 22 is presented in an easily understandable manner.

Specifically, the acquisition unit 50 acquires the three-dimensional model 28 showing the surface shape of the target part inside the body. The three-dimensional model 28 is a three-dimensional image including at least depth information of the surface of the target part. In addition, it is preferable that the acquisition unit 50 acquires the three-dimensional model 28 having real-time performance, which reflects the current state of the target part, over time. A method of acquiring (generating) the three-dimensional model 28 having real-time performance will be described below.

The display control unit 54 controls the display of the three-dimensional superimposed image 24 that is obtained by superimposing the internal image (for example, the ultrasound image 22) on the three-dimensional model 28 and in which a display aspect of the internal image is adjusted based on the position and orientation information. Specifically, the display control unit 54 generates the three-dimensional superimposed image 24 showing an imaging range (for example, the position, the orientation, and the size) of the ultrasound image 22 in the surgical field SF by superimposing the ultrasound image 22 on a region on the three-dimensional model 28 corresponding to the imaging range of the ultrasound image 22. The three-dimensional superimposed image 24 is an example of a “first superimposed image” of the present disclosure.

In addition, the display control unit 54 may perform control of increasing the transparency of a portion of the three-dimensional model 28 included in the three-dimensional superimposed image 24, the portion being located between the viewpoint of the three-dimensional superimposed image 24 and the display surface of the internal image. Increasing the transparency means making the portion of the three-dimensional model 28 translucent or transparent. FIG. 11 shows an example in which the three-dimensional model 28 between the viewpoint of the three-dimensional superimposed image 24 and the display surface (Xpb-Ypb plane) of the ultrasound image 22 is transparent.

That is, the three-dimensional superimposed image 24 in this case shows a cross section 28U of the three-dimensional model 28 in the same plane as the display surface (Xpb-Ypb plane) of the ultrasound image 22. According to such a form, it is possible to present a positional relationship in the depth direction between the three-dimensional model 28 and the ultrasound image 22 in a more easily understandable manner. In a case in which the three-dimensional model 28 represents only the surface shape, the inside of the cross section 28U is hollow.

In addition, the display control unit 54 may perform control of switching between displaying the three-dimensional superimposed image 24 based on the three-dimensional model 28 and displaying the superimposed image 26 based on the surgical field image 21 on the display 16. For example, the display control unit 54 may perform control of displaying the three-dimensional superimposed image 24 in a case in which a preset condition related to the display of the three-dimensional superimposed image 24 is satisfied, and may perform control of displaying the superimposed image 26 instead of the three-dimensional superimposed image 24 in a case in which the condition is not satisfied.

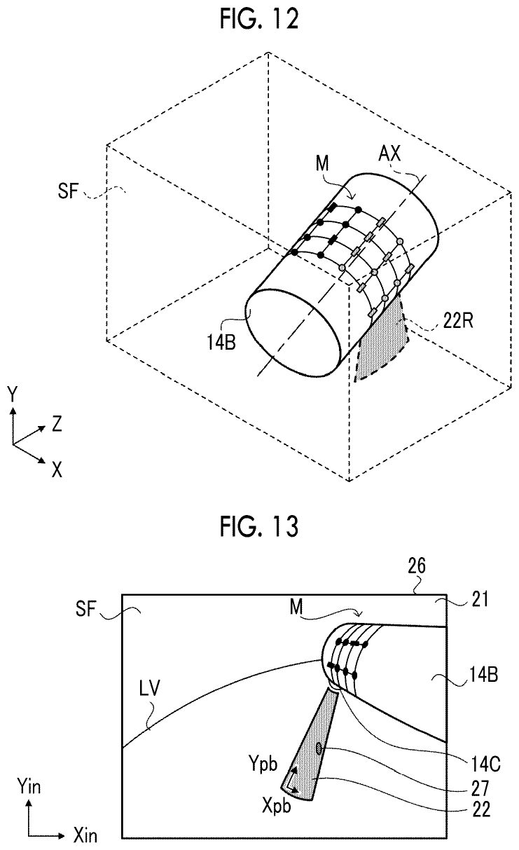

FIG. 12 shows a state in which the direction of the axis AX of the distal end part 14B of the ultrasound probe 14 is close to being parallel to the imaging optical axis of the camera 13B in the surgical field SF of the three-dimensional space. In this case, the imaging range (Xpb-Ypb plane) of the ultrasound image 22 in the surgical field SF and the Xin-Yin plane of the surgical field image 21 are close to being orthogonal to each other. Therefore, as shown in FIG. 13, the ultrasound image 22 superimposed on the surgical field image 21 has a form close to a linear shape, making it difficult to observe. In addition, in such a superimposed image 26, both the ultrasound image 22 and the surgical field image 21 are difficult to observe. The illustration method of FIG. 12 is the same as that of FIG. 6, and the illustration method of FIG. 13 is the same as that of FIG. 7.

That is, the smaller the angle θ1 is, which represents the direction of the internal image and is at its minimum in a case in which the imaging optical axis of the camera 13B is parallel to the display surface of the internal image and at its maximum in a case in which the imaging optical axis is a normal line, the more difficult it becomes to observe the superimposed image 26. Therefore, in a case in which the angle θ1 is less than a predetermined threshold value, the display control unit 54 may determine that a preset condition related to the display of the three-dimensional superimposed image 24 is satisfied. That is, in a case in which the angle θ1 is small and the superimposed image 26 is difficult to observe, the superimposed image 26 may be switched to the three-dimensional superimposed image 24 and displayed.

In addition, for example, the display control unit 54 may determine that the condition is satisfied in a case in which a user gives an instruction to display the three-dimensional superimposed image 24. That is, whether to display the three-dimensional superimposed image 24 or the superimposed image 26 may be switched according to the instruction from the user.

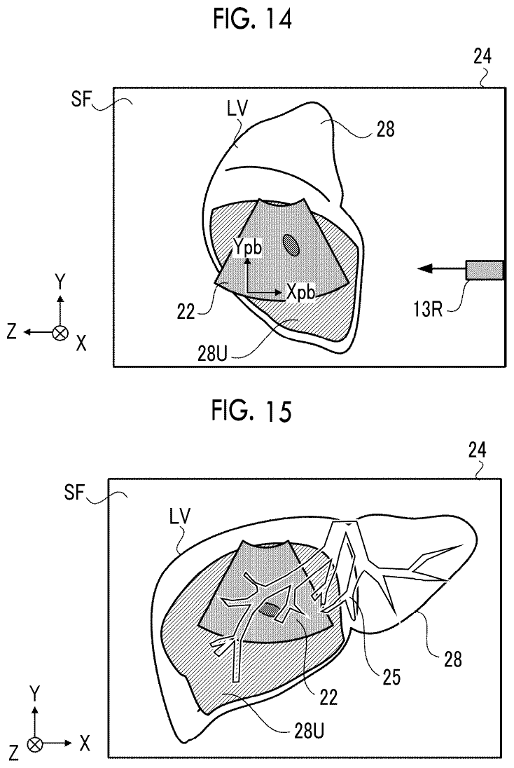

In addition, the display control unit 54 may receive designation of the direction of the three-dimensional model 28 in the three-dimensional superimposed image 24 and may generate the three-dimensional superimposed image 24 based on the designated direction of the three-dimensional model 28 and the position and orientation information. FIG. 14 shows the three-dimensional superimposed image 24 in a case in which the direction of the three-dimensional model 28 is rotated by 90 degrees about the Y-axis from the state of FIG. 10. As described above, the direction of the three-dimensional model 28 (that is, the viewpoint of the three-dimensional superimposed image 24) can be arbitrarily changed. Therefore, even though the ultrasound image 22 (see FIG. 13) is oriented in a direction that is difficult to observe in a case of being aligned with the viewpoint of the surgical field image 21, it can be displayed in a direction that is easy to observe while ensuring consistency with the three-dimensional model 28.

The direction of the three-dimensional model 28 may be arbitrarily specified by the user, for example. In addition, for example, a viewpoint that is shifted by 90 degrees about the Y-axis from the viewpoint of the surgical field image 21 may be designated in advance.

In addition, for example, the display control unit 54 may designate the direction of the three-dimensional model 28 such that the internal image superimposed on the three-dimensional model 28 faces the front. Specifically, in a case in which the viewpoint of the first superimposed image is located on a line perpendicular to the display surface of the internal image, the direction of the internal image is defined as being oriented to the front. In this case, the display control unit 54 may designate the direction of the three-dimensional model 28 such that the direction of the internal image determined based on the position and orientation information faces the front.

In addition, the display control unit 54 may perform control of displaying information indicating the position of the camera 13B (that is, the viewpoint of the surgical field image 21) on the three-dimensional superimposed image 24. FIG. 14 shows a rectangle 13R with an arrow attached along the imaging optical axis of the camera 13B as an example of the information indicating the position of the camera 13B. The information indicating the position of the camera 13B is not limited to this and may be indicated by, for example, text. According to such a form, even in a case in which the direction of the three-dimensional model 28 is changed in the three-dimensional superimposed image 24, the direction of the camera 13B actually inserted into the body is known, so that it is easy to adjust the position and the orientation of the endoscope 13, the ultrasound probe 14, and the like.

In addition, the display control unit 54 may apply, as the internal image to be superimposed on the three-dimensional superimposed image 24, an internal image represented in another way in addition to or instead of the ultrasound image 22. For example, the display control unit 54 may apply a tomographic image of the target part, which is extracted from a three-dimensional image captured by a tomographic imaging apparatus such as a computed tomography (CT) apparatus and a magnetic resonance imaging (MRI) apparatus, as the internal image. In addition, for example, the display control unit 54 may apply a blood vessel image indicating a blood vessel structure of the target part, which is extracted from a three-dimensional image captured by a tomographic imaging apparatus such as a CT apparatus, as the internal image. The tomographic image and the blood vessel image are examples of an “internal image” and a “second internal image” of the present disclosure.

Specifically, a three-dimensional image of the patient PT is captured in advance by a CT apparatus or the like and stored in the storage 43 or the like before the endoscopic surgery using the endoscope 13, the ultrasound probe 14, and the like. The acquisition unit 50 acquires the three-dimensional image including the internal structure of the target part, which is captured in advance. The display control unit 54 extracts the internal image from the three-dimensional image based on the position and orientation information.

For example, the display control unit 54 may extract the tomographic image and/or the blood vessel image corresponding to the display surface of the ultrasound image 22 from the three-dimensional image based on the position and orientation information. That is, the extracted tomographic image may be a tomographic image of the target part including the display surface of the ultrasound image 22. In addition, the extracted blood vessel image may be a blood vessel image showing a blood vessel structure of the target part passing through the display surface of the ultrasound image 22.

The display control unit 54 generates the three-dimensional superimposed image 24 using the internal image (tomographic image and/or blood vessel image) extracted from the three-dimensional image. For example, the display control unit 54 may generate the three-dimensional superimposed image 24 by superimposing the ultrasound image 22 and the internal image extracted from the three-dimensional image on the three-dimensional model 28.

FIG. 15 shows an example of a three-dimensional superimposed image 24 obtained by superimposing the blood vessel image 25 on the three-dimensional model 28 in addition to the ultrasound image 22. In this way, in a case in which a plurality of internal images are superimposed, the display control unit 54 may combine each internal image at a specific superimposition ratio. According to such a form, it is possible to check the internal image based on the CT image or the like even during a surgery, so that it is easier to grasp the internal structure of the target part. Therefore, convenience can be improved.

The display control unit 54 performs control of displaying at least one of the superimposed image 26 or the three-dimensional superimposed image 24 on the display 16. In addition, the display control unit 54 may perform control of displaying the surgical field image 21 and the ultrasound image 22 on the display 16. In this case, the display control unit 54 may perform control of displaying the surgical field image 21, the ultrasound image 22, the superimposed image 26, and the three-dimensional superimposed image 24 all together on one display 16.

In addition, in a case in which there are a plurality of the displays 16, the display control unit 54 may perform control of displaying the surgical field image 21, the ultrasound image 22, the superimposed image 26, and the three-dimensional superimposed image 24 on different displays 16. In addition, the display control unit 54 may receive designation of an image to be displayed on the display 16 among the surgical field image 21, the ultrasound image 22, the superimposed image 26, and the three-dimensional superimposed image 24, and may perform control of displaying the designated image on at least one display 16.

In addition, the surgical field image 21 and the ultrasound image 22 are output as video images. The display control unit 54 may perform control of displaying at least one of the surgical field image 21, the ultrasound image 22, the superimposed image 26, or the three-dimensional superimposed image 24 on the display 16 in a live view. The live view display refers to displaying an image generated at a predetermined frame rate based on a signal output by an imaging device for imaging a target, as a video image in real time. The imaging device includes, for example, an image sensor that optically images a target included in the endoscope 13, and the ultrasound transducer 14C that images the target using ultrasound waves.

In a case in which the superimposed image 26 and/or the three-dimensional superimposed image 24 is displayed in a live view, the derivation unit 52 repeats the derivation of the position and orientation information each time the surgical field image 21 is acquired or at a predetermined time interval. In response to this, the display control unit 54 repeats the generation of the superimposed image 26 and/or the three-dimensional superimposed image 24 such that the change in the position and orientation information is reflected.

### Generation of Three-Dimensional Model

It is preferable that the three-dimensional model 28 used for the three-dimensional superimposed image 24 reflects the current state of the target part. Therefore, the generation unit 56 generates the three-dimensional model 28 in real time by imaging the target part during the endoscopic surgery.

As an example, the generation unit 56 may generate (acquire) the three-dimensional model 28 using a stereo camera. The stereo camera is a camera that simultaneously captures an image of an imaging object in two different directions and that measures a distance to the imaging target from the parallax of each image.

Specifically, the generation unit 56 acquires the first image and the second image by optically imaging the surgical field SF including the target part (for example, the liver LV) inside the body from different viewpoints using the first camera and the second camera. For example, the first camera may be provided on the trocar 17 for inserting the medical instrument into the body. In addition, for example, the first camera may be provided on a body wall inside the body. In addition, for example, the second camera may be the camera 13B provided at the distal end part of the insertion part 13A of the endoscope 13 used in the endoscopic surgery.

In addition, the generation unit 56 detects a marker composed of an optically detectable pattern disposed in the surgical field SF from at least the first image. Then, the generation unit 56 specifies a relative positional relationship between the viewpoint of the first camera and the viewpoint of the second camera based on the detected marker. In the method using the stereo camera, it is necessary to specify a positional relationship between two cameras in order to obtain the parallax. This marker is provided at a position where at least the first camera can capture an image, as a reference for specifying a positional relationship between the two cameras.

Then, the generation unit 56 generates the three-dimensional model 28 showing the surface shape of the target part based on the specified relative positional relationship between the viewpoint of the first camera and the viewpoint of the second camera, the first image, and the second image. Hereinafter, a specific method for generating the three-dimensional model 28 using the generation unit 56 will be described with reference to examples.

### First Example

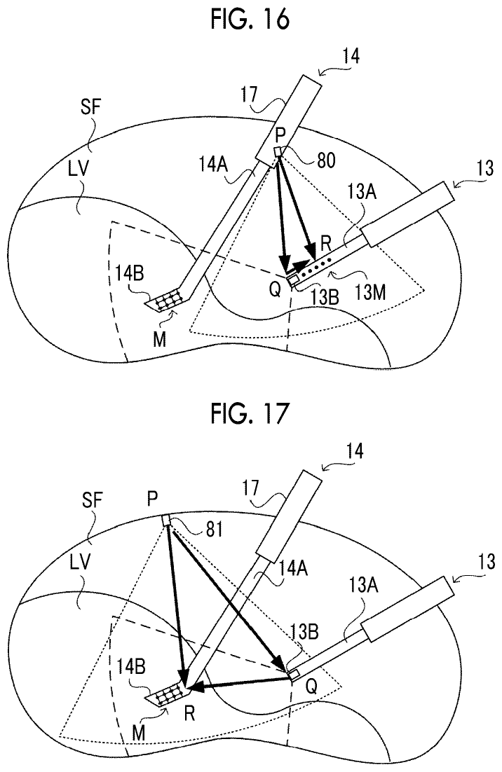

An example of a configuration for generating the three-dimensional model 28 using the stereo camera will be described with reference to FIG. 16. FIG. 16 shows a state in which the endoscope 13 and the ultrasound probe 14 are inserted into the abdomen of the patient PT. In this example, a camera 80 is provided on the trocar 17 for inserting the ultrasound probe 14 into the body as the first camera. A camera 13B provided at the distal end part of the insertion part 13A of the endoscope 13 is used as the second camera. In FIG. 16, a viewpoint of the camera 80 is denoted by P, and an imaging range of the camera 80 is indicated by a dotted line. In addition, a viewpoint of the camera 13B is denoted by Q, and an imaging range of the camera 13B is indicated by a broken line.

A marker 13M is attached to an outer peripheral surface of the camera 13B (more specifically, the distal end part of the insertion part 13A of the endoscope 13) as a reference marker for specifying a relative positional relationship (that is, a vector PQ) between the viewpoint P of the camera 80 and the viewpoint Q of the camera 13B. The marker 13M is a marker that can be recognized from the first image captured by the camera 80, that is, an optically detectable marker. A positional relationship between the viewpoint Q of the camera 13B and a position R of the marker 13M is known and is stored in the storage 43 in advance as, for example, the dimension information 45. In FIG. 16, the marker 13M is represented by five points, but the form of the marker 13M is not particularly limited.

The generation unit 56 specifies the relative positional relationship between the viewpoint P of the camera 80 and the viewpoint Q of the camera 13B based on the marker 13M in the first image captured by the camera 80. Specifically, the generation unit 56 first calculates a relative positional relationship (that is, a vector PR) between the viewpoint P of the camera 80 and the position R of the marker 13M based on the position and the form of the marker 13M in the first image. As described above, the positional relationship (that is, a vector QR) between the viewpoint Q of the camera 13B and the position R of the marker 13M is known. The generation unit 56 can calculate the vector PQ, which is a relative positional relationship between the viewpoint P of the camera 80 and the viewpoint Q of the camera 13B, from a difference between the vector PR and the vector QR.

The generation unit 56 generates the three-dimensional model 28 showing the surface shape of the target part (for example, the liver LV) based on the relative positional relationship between the viewpoint P of the camera 80 and the viewpoint Q of the camera 13B, the first image, and the second image. A technology using a known stereo method can be appropriately applied as a method of generating the three-dimensional model 28 using the first image and the second image.

In this example, an example in which the camera 80, which is the first camera, is provided on the trocar 17 has been described, but the present disclosure is not limited thereto. The camera 80 need only be provided at a position where the marker 13M can be imaged, and may be provided, for example, on a body wall or the like inside the body.

### Second Example

Another example of a configuration for generating the three-dimensional model 28 using the stereo camera will be described with reference to FIG. 17. FIG. 17 shows a state in which the endoscope 13 and the ultrasound probe 14 are inserted into the abdomen of the patient PT. In this example, a camera 81 is provided on the body wall inside the body as the first camera. A camera 13B provided at the distal end part of the insertion part 13A of the endoscope 13 is used as the second camera. In FIG. 17, a viewpoint of the camera 81 is denoted by P, and an imaging range of the camera 81 is indicated by a dotted line. In addition, a viewpoint of the camera 13B is denoted by Q, and an imaging range of the camera 13B is indicated by a broken line.

A marker M is attached to a medical instrument (ultrasound probe 14) inserted into the body as a reference marker for specifying a relative positional relationship (that is, a vector PQ) between the viewpoint P of the camera 81 and the viewpoint Q of the camera 13B. The marker M is imaged by each of the camera 81 and the camera 13B. As described above, the marker M is an optically detectable marker.

The generation unit 56 specifies a relative positional relationship between the viewpoint P of the camera 81 and the viewpoint Q of the camera 13B based on the marker M in the first image captured by the camera 81 and the marker M in the second image captured by the camera 13B. Specifically, first, the generation unit 56 calculates a relative positional relationship (that is, a vector PR) between the viewpoint P of the camera 81 and the position R of the marker M based on the position and the form of the marker M in the first image. Next, the generation unit 56 calculates a relative positional relationship (that is, a vector QR) between the viewpoint Q of the camera 13B and the position R of the marker M based on the position and the form of the marker M in the second image. The generation unit 56 can calculate the vector PQ, which is a relative positional relationship between the viewpoint P of the camera 81 and the viewpoint Q of the camera 13B, from a difference between the vector PR and the vector QR.

The generation unit 56 generates the three-dimensional model 28 showing the surface shape of the target part (for example, the liver LV) based on the relative positional relationship between the viewpoint P of the camera 81 and the viewpoint Q of the camera 13B, the first image, and the second image. A technology using a known stereo method can be appropriately applied as a method of generating the three-dimensional model 28 using the first image and the second image.

In this example, an example in which the marker is attached to the ultrasound probe 14 has been described, but the present disclosure is not limited thereto. The marker need only be provided at a position where the marker can be imaged by each of the camera 81 and the camera 13B, and may be provided, for example, on a treatment tool or the like that is separately inserted into the body.

The generation unit 56 is not limited to the method using the stereo camera, and may generate the three-dimensional model 28 using another method.

For example, the generation unit 56 may generate (acquire) the three-dimensional model 28 using a time of flight (ToF) camera. The ToF camera is a camera that irradiates the imaging target with light such as infrared rays and that measures a distance between the ToF camera and the imaging target based on a time until reflected light is received or a phase change between the emitted light and the received light. Specifically, an image captured by the ToF camera has distance information indicating a distance between the ToF camera and the imaging target for each pixel.

Therefore, for example, the three-dimensional model 28 showing the surface shape of the target part as viewed from the ToF camera may be generated by inserting the ToF camera into the body together with the endoscope 13, the ultrasound probe 14, and the like. The ToF camera may be provided on, for example, the trocar 17 or the like used for insertion of the endoscope 13 or the ultrasound probe 14. In addition, the acquisition unit 50 may generate the three-dimensional model 28 by combining the ToF camera and a visible light camera (for example, the camera 13B of the endoscope 13).

In addition, for example, the acquisition unit 50 may acquire the three-dimensional model 28 based on a three-dimensional image captured by a tomographic imaging apparatus such as a CT apparatus and an MRI apparatus. In this case, for example, before the endoscopic surgery, a three-dimensional image of the patient PT may be captured in advance and stored in the storage 43 or the like. The acquisition unit 50 may acquire the three-dimensional model 28 by acquiring the three-dimensional image that has been captured in advance and applying known three-dimensional reconstruction processing. In this case, it is difficult to generate the three-dimensional model 28 in real time, but it is possible to generate a more accurate three-dimensional model 28 including the internal structure.

In addition, for example, the generation unit 56 may generate the three-dimensional model 28 by combining the above-described methods. For example, in the method using the stereo camera, it may not be possible to capture an image of the back side of the target part as viewed from the viewpoint of the camera, and a part of the three-dimensional model 28 may be missing. Therefore, for example, the generation unit 56 may basically generate the three-dimensional model 28 in real time using the stereo camera and reconstruct a portion that cannot be generated by the stereo camera from the three-dimensional image that has been captured in advance.

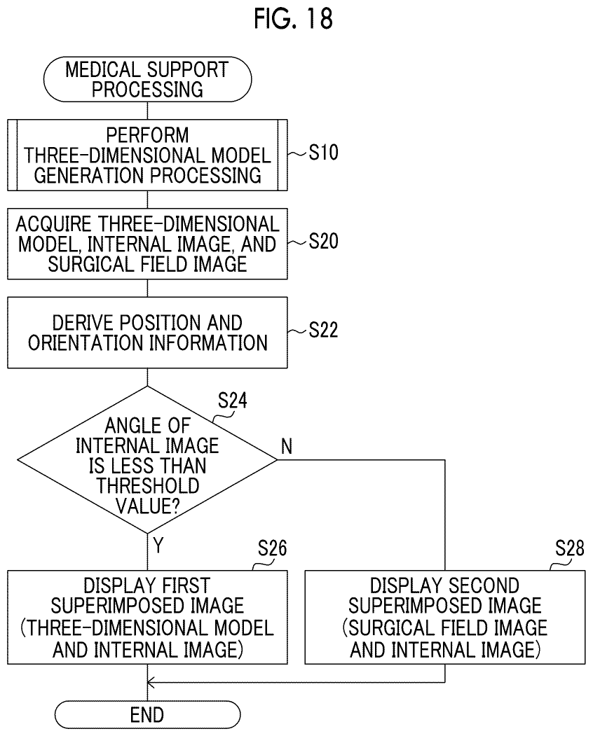

Next, an operation of the medical support device 11 according to the present embodiment will be described with reference to FIG. 18. In the medical support device 11, the medical support processing shown in FIG. 18 is executed by the processor 41 executing the medical support program 44. This processing is executed, for example, in a case in which a user gives an instruction to start execution via the reception device 46.

In step S10, the generation unit 56 performs processing of generating the three-dimensional model showing the surface shape of the target part inside the body. The three-dimensional model generation processing will be described below.

In step S20, the acquisition unit 50 acquires the three-dimensional model generated in step S10. In addition, the acquisition unit 50 acquires an internal image showing the internal structure of the target part. In addition, the acquisition unit 50 acquires the surgical field image obtained by optically imaging the surgical field SF including the target part inside the body and the medical instrument inserted into the body using the camera.

In step S22, the derivation unit 52 acquires the position and orientation information indicating the position and the orientation of the medical instrument in the surgical field SF. In step S24, the display control unit 54 determines whether or not an angle θ1, which is a direction of the internal image acquired in step S20, is less than a predetermined threshold value. The angle θ1 is at its minimum in a case in which the imaging optical axis of the camera 13B is parallel to the display surface of the internal image and is at its maximum in a case in which the imaging optical axis is a normal line. Specifically, the display control unit 54 derives the angle θ1 based on the position and orientation information derived in step S12 and the dimension information 45.

In a case in which the angle θ1 is less than the threshold value and the result of step S24 is positive, the processing proceeds to step S26. In step S26, the display control unit 54 performs control of displaying, on the display 16, the first superimposed image that is obtained by superimposing the internal image on the three-dimensional model acquired in step S20 and in which the display aspect of the internal image is adjusted based on the position and orientation information derived in step S22.

On the other hand, in a case in which the angle θ1 is equal to or greater than the threshold value and the result of step S24 is negative, the processing proceeds to step S28. In step S28, the display control unit 54 performs control of displaying, on the display 16, the second superimposed image that is obtained by superimposing the internal image on the surgical field image acquired in step S20 and in which the display aspect of the internal image is adjusted based on the position and orientation information derived in step S22. In a case in which step S26 or step S28 is completed, this processing is ended.

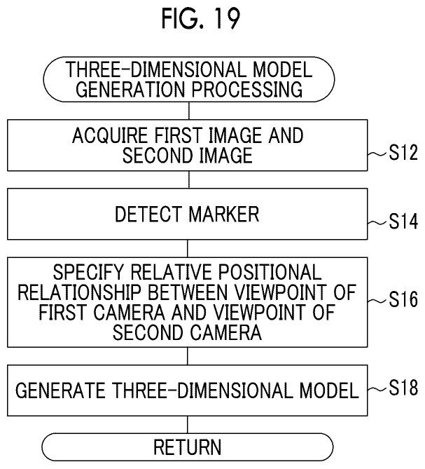

Next, the three-dimensional model generation processing executed in step S10 will be described with reference to FIG. 19. In step S12, the generation unit 56 acquires the first image and the second image obtained by optically imaging the surgical field SF including the target part inside the body from different viewpoints using the first camera and the second camera. In step S14, the generation unit 56 detects a marker composed of an optically detectable pattern disposed in the surgical field SF from at least the first image.

In step S16, the generation unit 56 specifies a relative positional relationship between the viewpoint of the first camera and the viewpoint of the second camera based on the marker detected in step S14. In step S18, the generation unit 56 generates the three-dimensional model 28 showing the surface shape of the target part based on the relative positional relationship between the viewpoint of the first camera and the viewpoint of the second camera specified in step S16 and the first image and the second image acquired in step S12.

As described above, the medical support device 11 according to one aspect of the present embodiment comprises the processor 41. The processor 41 acquires the three-dimensional model showing the surface shape of the target part inside the body and acquires the internal image showing the internal structure of the target part. In addition, the processor 41 acquires the position and orientation information indicating the position and the orientation of the medical instrument in the surgical field SF including the target part and the medical instrument inserted into the body. In addition, the processor 41 controls the display of the first superimposed image that is obtained by superimposing the internal image on the three-dimensional model and in which the display aspect of the internal image is adjusted based on the position and orientation information.

With the medical support device 11 according to the present embodiment, it is possible to generate the three-dimensional superimposed image 24 by superimposing the internal image on the three-dimensional model 28. Therefore, it is possible to present the positional relationship between the target part and the imaging range of the internal image in the surgical field SF in an easily understandable manner, and to support the observation of the internal image.

In addition, the medical support device 11 according to another aspect of the present embodiment comprises the processor 41. The processor 41 acquires the first image and the second image obtained by optically imaging the surgical field including the target part inside the body from different viewpoints using the first camera and the second camera. In addition, the processor 41 detects a marker composed of an optically detectable pattern disposed in the surgical field SF from at least the first image. In addition, the processor 41 generates the three-dimensional model showing the surface shape of the target part based on the relative positional relationship between the viewpoint of the first camera and the viewpoint of the second camera, which is specified based on the marker, the first image, and the second image.

With the medical support device 11 according to the present embodiment, a positional relationship between two cameras of which the positional relationship is not known can be easily specified even in a body cavity, so that it is easy to generate the three-dimensional model 28 in real time using the stereo method. In addition, in the stereo method, in a case in which the parallax is too small, the accuracy of the three-dimensional model 28 tends to decrease. However, with the medical support device 11 according to the present embodiment, the degree of freedom of positions of the two cameras can be ensured, so that the decrease in the accuracy of the three-dimensional model 28 can be avoided. That is, the three-dimensional model 28 having excellent real-time performance and accuracy can be generated, and, in a case of being applied to the three-dimensional superimposed image 24, it can support in observing the internal image.

In the above-described embodiment, the form in which the distal end part 14B of the insertion part 14A of the ultrasound probe 14 is provided with the marker M has been described, but the present disclosure is not limited to this. The marker M need only be provided to a portion of various medical instruments that is inserted into the body of the patient PT, and may be provided, for example, to a middle portion of the insertion part 14A and a base end side of the insertion part 14A.

In addition, in the above-described embodiment, the form in which the position and orientation information indicating the position and the orientation of the medical instrument in the surgical field SF is derived using the marker M has been described, but the present disclosure is not limited to this. For example, the position and orientation information may be derived by detecting a characteristic shape of the medical instrument from the surgical field image 21 through image analysis. In addition, for example, a shape of the medical instrument may be specified by imaging the medical instrument using a ToF camera or a stereo camera, and the position and orientation information may be derived based on the shape.

In addition, in the above-described embodiment, the form in which the camera 13B of the endoscope 13 captures the surgical field image 21 has been described, but the present disclosure is not limited to this. For example, an image captured by a camera provided on another instrument inserted into the body, such as the trocar 17, may be used, or an image captured by a camera provided on the body wall may be used.

In addition, in the above-described embodiment, the medical instrument to be inserted into the body of the patient PT is the ultrasound probe 14 (an example of a medical probe) that can observe the internal structure of the organ. The treatment of puncturing the organ inside the body with the biopsy needle 18 is often performed using the medical probe capable of observing the internal structure of the organ. Therefore, as in the above-described embodiment, the technology of the present disclosure is particularly effective in a case in which the medical probe is used as the medical instrument.

Further, the ultrasound probe 14 is used in combination with the biopsy needle 18 relatively frequently. Therefore, the technology of the present disclosure is more effective in a case in which the ultrasound probe 14 is used as the medical probe. As the medical probe capable of observing the internal structure of the organ, a probe other than the ultrasound probe 14 may be used, such as an optical coherence tomography (OCT) probe. In addition, for example, a drop-in type probe may be used.

As the medical instrument, a medical instrument other than the medical probe capable of observing the internal structure of the organ can also be applied. For example, as the medical instrument, a treatment tool may be used, which does not have an internal structure observation function and has only the guide groove 29 of the biopsy needle 18 at the distal end part. For example, in a case in which the tumor exists on the surface of the organ, in a case in which the treatment of puncturing the tumor on the surface with the biopsy needle 18 is performed, even with the treatment tool that does not have the internal structure observation function, the biopsy needle 18 can be appropriately guided as long as the guide groove 29 is provided. In this case, for example, the medical staff ST positions the guide groove 29 of the treatment tool at a position of the surface of the organ corresponding to the tumor, and in this state, the medical staff ST punctures the tumor with the biopsy needle 18 through the guide groove 29.

Further, as the medical instrument, a treatment tool such as a simple rod without including the guide groove 29 may be used. In a case in which the tumor exists on the surface of the organ, it is possible to point to the tumor even with such a treatment tool. Even in a case in which a puncture path 30 is simply superimposed and displayed on the surgical field image 21 in which the treatment tool points to the tumor on the surface of the organ, the puncture path 30 serves as a guide for checking a puncture direction of the biopsy needle 18 or the like. Therefore, the technology of the present disclosure is effective even for a medical instrument that is a treatment tool without including the guide groove 29.

In addition, in the above-described embodiment, the cauterization has been described as an example of the function of the biopsy needle 18, but the function of the biopsy needle 18 is not limited to this. In addition, although the biopsy needle 18 has been described as an example of the treatment tool, a treatment tool for injecting a fluorescent agent such as indocyanine green (ICG), a biopsy needle used for tissue collection for performing a biopsy, forceps, and the like may be applied instead of the biopsy needle 18.

In addition, in the above-described embodiment, the body cavity such as the abdominal cavity and the thoracic cavity has been described as the inside of the body, but the inside of the body may be an upper digestive tract such as an esophagus, a lower digestive tract such as an intestine, and a tubular organ such as a bronchus. In a case in which the technology of the present disclosure is applied to a surgical field in the tubular organ, for example, the marker M is provided in a base end part of a soft endoscope to be inserted into the tubular organ.

In addition, in the above-described embodiment, the example in which the medical support device 11 is used in the endoscopic surgery has been described, but the present disclosure is not limited to this. The medical support device 11 can be applied to, for example, a robot surgery.

In addition, in the above-described embodiment, for example, as hardware structures of processing units that execute various kinds of processing, such as the acquisition unit 50, the derivation unit 52, the display control unit 54, and the generation unit 56, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured of one of the various processors, or may be configured of a combination of the same or different kinds of two or more processors (for example, a combination of a plurality of FPGAs or a combination of the CPU and the FPGA). In addition, a plurality of processing units may be configured of one processor.

As an example in which a plurality of processing units are configured of one processor, first, as typified by a computer such as a client or a server, there is an aspect in which one processor is configured of a combination of one or more CPUs and software, and this processor functions as a plurality of processing units. Second, as typified by a system on chip (SoC) or the like, a processor that realizes the functions of the entire system including the plurality of processing units by using one integrated circuit (IC) chip is used. As described above, various processing units are configured by using one or more of the various processors as a hardware structure.

Furthermore, as the hardware structure of these various processors, more specifically, an electric circuit (circuitry) in which circuit elements such as semiconductor elements are combined can be used.

In addition, in the above-described embodiment, the medical support program 44 has been described as being stored (installed) in the storage 43 in advance, but the present disclosure is not limited to this. The medical support program 44 may be provided in a form of being recorded on a recording medium, such as a compact disc read only memory (CD-ROM), a digital versatile disc read only memory (DVD-ROM), or a universal serial bus (USB) memory. In addition, the medical support program 44 may be downloaded from an external device via a network.

In addition, the present disclosure can also be applied to a program and a program product. Specifically, the medical support program 44 in the above-described embodiment may be provided as a program product. The program product includes a product in any aspect for providing a program. For example, the program product includes a program provided through a network such as the Internet, and a computer-readable recording medium that non-transitorily stores the program.

In the technology of the present disclosure, the embodiment and the modification examples described above can be combined as appropriate. The content of the above description and the content of the drawings are detailed explanations of the parts relating to the technology of the present disclosure, and are merely examples of the technology of the present disclosure. For example, description related to the above configurations, functions, actions, and effects is description related to an example of configurations, functions, actions, and effects of the parts according to the embodiments of the technology of the present disclosure. As a result, it goes without saying that unnecessary parts may be deleted, new elements may be added, or replacements may be made with respect to the above-described contents and the above-shown contents within a range that does not deviate from the gist of the technology of the present disclosure.

The following appendices are disclosed with regard to the above embodiment.

### APPENDIX 1

A medical support device comprising:
a processor,
in which the processor is configured to
   acquire a first image and a second image by optically imaging a surgical field including a target part inside a body from different viewpoints using a first camera and a second camera,
   detect a marker composed of an optically detectable pattern disposed in the surgical field from at least the first image, and
   generate a three-dimensional model showing a surface shape of the target part based on a relative positional relationship between the viewpoint of the first camera and the viewpoint of the second camera, the first image, and the second image, the relative positional relationship being specified based on the marker.

### APPENDIX 2

The medical support device according to Appendix 1,
in which the first camera is a camera provided on a trocar for inserting a medical instrument into the body or on a body wall inside the body.

### APPENDIX 3

The medical support device according to Appendix 1 or 2,
in which the second camera is a camera provided at a distal end part of an endoscope used in an endoscopic surgery.

### APPENDIX 4

The medical support device according to any one of Appendices 1 to 3,
in which the marker is attached to an outer peripheral surface of the second camera and is imaged by the first camera, and
the processor is configured to specify the relative positional relationship between the viewpoint of the first camera and the viewpoint of the second camera based on the marker in the first image.

### APPENDIX 5

The medical support device according to any one of Appendices 1 to 3,
in which the marker is attached to a medical instrument to be inserted into the body and is imaged by each of the first camera and the second camera, and
the processor is configured to specify the relative positional relationship between the viewpoint of the first camera and the viewpoint of the second camera based on the marker in the first image and the marker in the second image.

### APPENDIX 6

The medical support device according to Appendix 5,
in which the medical instrument is an ultrasound probe that transmits an ultrasound wave to the target part and that detects an electric signal corresponding to an ultrasound echo reflected from the target part.

### APPENDIX 7

The medical support device according to any one of Appendices 1 to 6,
in which the processor is configured to
   acquire an internal image showing an internal structure of the target part,
   acquire position and orientation information indicating a position and an orientation of a medical instrument inserted into the body, and
   control display of a superimposed image that is obtained by superimposing the internal image on the three-dimensional model and in which a display aspect of the internal image is adjusted based on the position and orientation information.

### APPENDIX 8

A medical support method executed by a computer, the medical support method comprising:
acquiring a first image and a second image by optically imaging a surgical field including a target part inside a body from different viewpoints using a first camera and a second camera;
detecting a marker composed of an optically detectable pattern disposed in the surgical field from at least the first image; and
generating a three-dimensional model showing a surface shape of the target part based on a relative positional relationship between the viewpoint of the first camera and the viewpoint of the second camera, the first image, and the second image, the relative positional relationship being specified based on the marker.

### APPENDIX 9

A medical support program causing a computer to execute:
acquiring a first image and a second image by optically imaging a surgical field including a target part inside a body from different viewpoints using a first camera and a second camera;
detecting a marker composed of an optically detectable pattern disposed in the surgical field from at least the first image; and
generating a three-dimensional model showing a surface shape of the target part based on a relative positional relationship between the viewpoint of the first camera and the viewpoint of the second camera, the first image, and the second image, the relative positional relationship being specified based on the marker.

What is claimed is:

1. A medical support device comprising a processor, wherein the processor is configured to:
acquire a first image and a second image by optically imaging a surgical field including a target part inside a body from different viewpoints using a first camera and a second camera;
detect a marker composed of an optically detectable pattern disposed in the surgical field from at least the first image; and
generate a three-dimensional model showing a surface shape of the target part based on a relative positional relationship between the viewpoint of the first camera and the viewpoint of the second camera, the first image, and the second image, the relative positional relationship being specified based on the marker.

2. The medical support device according to claim 1, wherein the first camera is a camera provided on a trocar for inserting a medical instrument into the body or on a body wall inside the body.

3. The medical support device according to claim 1, wherein the second camera is a camera provided at a distal end part of an endoscope used in an endoscopic surgery.

4. The medical support device according to claim 1, wherein:
the marker is attached to an outer peripheral surface of the second camera and is imaged by the first camera, and
the processor is configured to specify the relative positional relationship between the viewpoint of the first camera and the viewpoint of the second camera based on the marker in the first image.

5. The medical support device according to claim 1, wherein:
the marker is attached to a medical instrument to be inserted into the body and is imaged by each of the first camera and the second camera, and
the processor is configured to specify the relative positional relationship between the viewpoint of the first camera and the viewpoint of the second camera based on the marker in the first image and the marker in the second image.

6. The medical support device according to claim 5, wherein the medical instrument is an ultrasound probe that transmits an ultrasound wave to the target part and that detects an electric signal corresponding to an ultrasound echo reflected from the target part.

7. The medical support device according to claim 1, wherein the processor is configured to:
acquire an internal image showing an internal structure of the target part;
acquire position and orientation information indicating a position and an orientation of a medical instrument inserted into the body; and
control display of a superimposed image that is obtained by superimposing the internal image on the three-dimensional model and in which a display aspect of the internal image is adjusted based on the position and orientation information.

8. A medical support method executed by a computer, the medical support method comprising:
acquiring a first image and a second image by optically imaging a surgical field including a target part inside a body from different viewpoints using a first camera and a second camera;
detecting a marker composed of an optically detectable pattern disposed in the surgical field from at least the first image; and
generating a three-dimensional model showing a surface shape of the target part based on a relative positional relationship between the viewpoint of the first camera and the viewpoint of the second camera, the first image, and the second image, the relative positional relationship being specified based on the marker.

9. A non-transitory computer-readable storage medium storing a medical support program causing a computer to execute a process comprising:
acquiring a first image and a second image by optically imaging a surgical field including a target part inside a body from different viewpoints using a first camera and a second camera;
detecting a marker composed of an optically detectable pattern disposed in the surgical field from at least the first image; and
generating a three-dimensional model showing a surface shape of the target part based on a relative positional relationship between the viewpoint of the first camera and the viewpoint of the second camera, the first image, and the second image, the relative positional relationship being specified based on the marker.
